(19) United States
(12) Patent Application Publication
(10) Pub. No.: US 2002/0103381 A1
(43) Pub. Date: Aug. 1, 2002

(54) Method for the synthesis of pyrazolines

(76) Inventor: Armen M. Boldi, Burlingame, CA (US)

Correspondence Address: Cooley Godward LLP, Attention: Patent Group, Five Palo Alto Square, 3000 El Camino Real, Palo Alto, CA, 94306-2155, US

(21) Appl. No.: 09/727,813

(22) Filed: Nov. 30, 2000

Related U.S. Application Data
(60) Provisional application No. 60/168,836, filed on Jan. 16, 2001.

(51) Int. Cl. C07D 403/02; G01N 33/53; G01N 33/543; C07D 231/06

(52) U.S. Cl. 548/379.4; 548/364.7; 435/7.1; 436/518

(57) ABSTRACT

The present invention provides a method for the synthesis of compounds of Formula I:

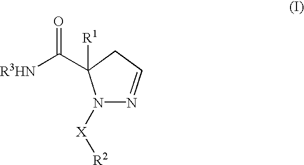

Also claimed are novel intermediates and their methods of synthesis.

### CROSS REFERENCE TO RELATED APPLICATIONS

This application claims the benefit of U.S. Provisional Application No. 60/168,836, filed on Dec. 3, 1999.

### FIELD OF INVENTION

The present invention relates to the synthesis of pyrazolines.

### BACKGROUND

The synthesis of pyrazoline derivatives has been reported by El-Rayyes, et al., Synthesis 1985, 1028-1042. Carreira and co-workers have reported the synthesis of pyrazoline based compounds by cycloaddition of trimethylsilyldiazomethane to α,β-unsaturated sulfonamides (Mish, et al., J. Am. Chem. Soc. 1997, 199, 8379-8380).

Pyrazoline based compounds have been known to have useful biological activity. Nugent et al. have reported the synthesis of pyrazoline based compounds as anti-inflammatory and antiarthritic agents in J. Med. Chem. 1993, 36:134-139. Pyrazoline based compounds have been reported to have cerebrovascular ischemia activity with an example, MS- 153, in Phase II clinical trials. Pyrazoline based compounds are thus an important class of compounds with applications in the pharmaceutical industry.

In spite of the different synthetic techniques known in the art, the pharmaceutical industry is in search of new processes that will enable synthesis of a large number of compounds at a relatively rapid pace. There is thus a need for a process for rapid parallel synthesis of multiple pyrazoline based compounds.

### SUMMARY OF THE INVENTION

The present invention provides a method for synthesizing a compound or a library of pyrazoline based compounds represented by Formula I.

### DETAILED DESCRIPTION

Definitions:

The following terms and phrases as used herein have the following meaning, unless indicated otherwise.

The term “array of compounds” or “library of compounds” indicates a collection of independent (individual) compounds that are synthesized by the method of the present invention. Generally the term array of compounds indicates a collection of individual compounds distinct from one another. Also included in the array (library) of compounds is a mixture of the individual compounds. The term “library of compounds” can be interchangeably used with the term “array of compounds”.

The term “alkyl” as used herein represents a straight (unbranched) or branched saturated hydrocarbon radical comprising from one to fourteen carbon atoms, unless indicated otherwise and includes as methyl, ethyl, n-propyl, isopropyl, n-butyl, isobutyl, sec-butyl, tert-butyl and the like, The term “(CH₂)ₙ” refers to a straight carbon chain linker having “n” carbons. When n is zero, the linker is a covalent bond.

The term “cycloalkyl” as used herein represents a saturated cyclic hydrocarbon radical comprising from four to ten carbon atoms, unless indicated otherwise.

The term “aryl” as used herein represents an aromatic hydrocarbon comprising from six to ten carbon atoms, unless indicated otherwise.

The term “heteroaryl” as used herein represents an aryl group having 1-3 heteroatoms within a single ring, (e.g., such as pyridyl, imidazolyl, thiazolyl, pyrimidine, oxazolyl, and the like), or within two rings (e.g., indolyl, quinolinyl, benzofuranyl, and the like).

The terms “optionally substituted aryl” and “optionally substituted heteroaryl” represents an aryl or heteroaryl group, as defined above, that can be substituted with one or more substituents selected from the group consisting of —OCF₃, halogen, —S—haloalkyl, —S—haloaryl, —NH(CH₂)₁₋₄—CN, —N[(CH₂)₁₋₄—CN)]₂, and —O—C(O)—C₁₋₄alkyl.

The term “optional” or “optionally” generally means that the subsequently described event or circumstance may or may not occur, and that the description includes instances where the event or circumstance occurs and instances in which it does not. For example, the phrase “optionally is substituted with one or more substituents”, as used above, means that the group referred to may or may not be substituted in order to fall within the scope of the invention. Thus the term “optionally substituted” is intended to mean that any one or more hydrogens on a designated atom can be replaced with a selection from the indicated group, provided that the designated atom's normal valence is not exceeded, and that the substitution results in a stable compound. When the substituent is keto (═O) then 2 hydrogens on the atom are replaced.

The term “alkoxy” represents an oxygen atom attached to an alkyl group and comprises from one to four carbon atoms, unless indicated otherwise.

The term “alkenyl” as used herein represents a straight (unbranched) or branched unsaturated hydrocarbon radical containing about 1 to 3 double bonds and 2 to 14 carbon atoms, unless indicated otherwise, and includes ethene, 1-propene, 1-butene, 3-methylbut-1-ene, 1 -pentene, and the like.

A “dehydrating agent” as used herein represents a reagent which absorbs or traps any water that is formed during a reaction. Illustrative examples of a dehydrating agent are dicyclohexylcarbodiimide and diisopropylcarbodiimide.

The term “deprotecting agent” represents a reagent which is capable of removing a protecting group. Illustrative examples of deprotecting agent are trifluoroacetic acid (TFA), trifluoromethanesulofonic acid, perfluorobutyric acid, sulfiric acid, hydrochloric acid, hydrofluoric acid and hydrobromic acid.

Illustrative examples of an “optional base” and “a suitable base” are N-methylmorpholine (NMM), N-ethylmorpholine, N-methylpiperidine, N-ethylpiperidine, triethylamine, pyridine, lutidine, diisopropylethylamine and N,N-diisopropylethylamine (DIPEA).

As used in the present invention, the term “solid support” or “SS” signifies a polymeric material for supported synthesis. The solid support should be labile under certain conditions to facilitate formation of a compound of Formula I, and should otherwise be chemically stable under the conditions of the present method. Illustrative examples of suitable solid supports are 3,5-dimethoxy-4-formylphenoxy polystyrene; 2-(3,5-dimethoxy-4-formylphenoxy)ethoxymethyl polystyrene; 2-(3,5-dimethoxy-4-formylphenoxy)ethyl polystyrene; 4-(3,5-dimethoxy-4-formylphenoxy)-butyramidomethyl polystyrene; 4-(3,5-dimethoxy-4-formylphenoxy) propionamidomethyl polystyrene; and 4-(3,5-dimethoxy-4-fornylphenoxy)acetamidomethyl. A detailed description of the terms linker molecule and solid support can be found in B. A. Bunin, “The Combinatorial Index”, Academic Press (1998), which is incorporated herein by reference.

The term “inert solvent” is intended to represent solvents which do not react with the reagents dissolved therein. Illustrative examples of inert solvents are tetrahydrofaran, methylene chloride, dichloromethane, ethyl acetate, dimethylformamide, diaoxane, chloroform and dimethylsulfoxide.

The term “catalyst” is intended to represent an additive that facilitates the course of a reaction but does not get incorporated in to the final product. Illustrative examples of catalysts are N-hydroxybenzotriazole, 1-hydroxy-7-azabenzotriazole and N-hydroxysuccinimide.

The term “protecting group” or “PG” is meant to represent a group which when attached to a reactive center renders the reactive center unreactive. An illustrative example of a protecting group is trialkyl silyl. A comprehensive list of suitable protecting groups can be found in T. W. Greene, “Protecting Groups in Organic Synthesis”, John Wiley & Sons, Inc. (1981), which is incorporated herein by reference.

The term “suitable primary amine substituent” as used herein represents a substituent which is capable of forming a covalent bond with an amine group to form a compound of Formula 9. Illustrative examples of suitable primary amine substituents are optionally substituted alkyl groups, optionally substituted aryl groups, optionally substituted heteroaryl groups, optionally substituted alkoxy groups, 2-hydroxy-1-hydroxymethyl-2-phenylethylamino; 2-hydroxy-1-hydroxymethyl-3-methylpentylamino; 1-benzyl-2-hydroxyethylamino; 1-hydroxymethyl-3-methylbutylamino; 4-amino-piperidine-1-carboxylic acid ethyl ester; 2-acetylaminoethylamino; 2-diethylaminoethylamino; 2-(2-hydroxy-ethoxy)-ethylamino; 3-diethylaminopropylamino; 3-hydroxypropylamino; 6-hydroxyhexylamino; 3-imidazol-1-yl-propylamino; 2-(4-sulfamoylphenyl)ethylamino; 3-(4-methylpiperazin-1-yl)-propylamino; 2-dimethylamino-l-methylethylamino; 2-[bis-(2-hydroxyethyl)amino] ethylamino;

1-carbamoyl-2-phenylethylamino; 2-dibutylaminoethylamino; 5-hydroxy-4,4-dimethylpentylamino; 3-dimethylamino-2,2-dimethylpropylamino; 2-(butylethylamino)ethylamino; 2-diisobutylaminoethylamino; 2-hydroxybutylamino; 3-hydroxy-2,2-dimethylpropylamino; cyclohexylamino; (5-hydroxy-1,3,3-trimethylcyclohexylmethyl)amino; 1,2,3,4-tetrahydronaphthalen-1-ylamino; cyclooctylamino; 3-(2-oxopyrrolidin-1-yl)propylamino; indan-1-ylamino, (tetrahydrofuran-2-ylmethyl)amino; 2-(1h-indol-3-yl)ethylamino; (benzo[1,3]dioxol-5-ylmethyl)amino; 3-morpholin-4-yl-propylamino; 2-pyridin-2-yl-ethylamino; 2-hydroxy-1-methyl-2-phenylethylamino; 1-methoxyethylamino; 1-methyl-3-phenylpropylamino; 3-diethylamino-1-methylpropylamino; benzylamino; 2-fluorobenzylamino; 2-methoxybenzylamino; 3-trifluoromethylbenzylamino; 2-phenylaminoethylamino; 2-methoxyethylamino; phenethylamino; 2-(2-methoxyhenyl)ethylamino; 2-(3,4-dimethoxy-phenyl)ethylamino; 2-(4-chlorophenyl)ethylamino; 2-(4-methoxyphenyl)ethylamino; 2-(4-hydroxyphenyl)ethylamino; 3,3-diphenylpropylamino; 2,5-dimethylbenzylamino; 2-trifluoromethylbenzylamino; butylarnino; 1 ,2-diethylpyrazolidin-4-ylamino; 3-methoxypropylamino; 2-diisopropylaminoethylamino; 1-isopropyl-2-methylpropylamino; 3-m-tolylaminopentylamino; 3-butoxypropylamino; 1-(4-fluorophenyl)ethylamino; 1-methoxymethylpropylamino; 2,3-dimethoxybenzylamino; 2,4-dimethoxybenzylamino; 2-(2-chloro-6-fluorobenzylsulfanyl)ethylamino; 2,6-dimethoxybenzylamino; 3,5-dimethoxybenzylamino; 2-phenoxyethylamino; 1-benzylpyrrolidin-3-ylamino; 2-(2,3-dimethoxyphenyl)ethylamino; 2-(2,5-dimethoxyphenyl)ethylamino; 2-(2-ethoxyphenyl) ethylamino; 2-(3,5-dimethoxyphenyl)ethylamino; 2-(4-ethoxyphenyl)ethylamino; 2-(4-trifluoromethoxyphenyl)ethylamino; 2-hydroxy-1,2-diphenylethylamino; 2-hydroxy-1,2-diphenylethylamino; 2-(2-hydroxymethylphenylsulfanyl)benzylamino; 2- (3-fluorophenyl) ethylamino; 2-(2-aminophenyl)benzylamino; 2-(2-fluorophenyl)ethylamino; 4-aminobenzylamino; 2-(3,4-dimethoxyphenyl)ethylamino; 1,2-dihydroxy-2-(4-methylsulfanylphenyl) ethylamino; 2-hydroxycyclohexylamino; and 3-(methylphenylamino) propylamino. A comprehensive list of amines substituted with a substituted with suitable substituents can be found in the Aldrich Chemicals catalog, which is incorporated herein by reference.

| | Abbreviations |
|---|---|
| DIC | Diisopropylcarbodiimide |
| DIPEA | N,N-diisopropylethylamine |
| DMAP | Dimethylaminopyridine |
| DMF | N,N-Dimethylformamide |
| DMSO | Dimethylsulfoxide |
| ESI | Electrospray ionization |
| Eschenmoser's Salt | Dimethyl methylene ammonium iodide |
| HOAt | 1-hydroxy-7-azabenzotriazole |
| HOBt | N-hydroxybenzotriazole |
| LC/MS | Liquid chromatography/mass spectroscopy |
| MS | Mass spectroscopy |
| NMM | N-Methylmorpholine |
| Ph | Phenyl |
| TEA | Triethylamine |
| TFA | Trifluoroacetic acid |
| THF | Tetrahydrofuran |
| TLC | Thin layer chromatography |

The present invention provides a method for synthesizing a compound or an array of compounds of Formula I:

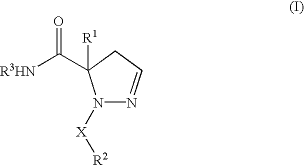

wherein:

R¹ is selected from the group consisting of C₁₋₁₄ alkyl, (CH₂)₀₋₆aryl, (CH₂)₀₋₂CH(Ph)₂, (CH₂)₁₋₄aryl(C₁₋₄alkoxy)₁₋₃, (CH₂)₀₋₄-Z—(CH₂)₁₋₄aryl-(C₁₋₄ alkoxy)₀₋₄, (CH₂)₁₋₄-Z-aryl-(CF₃)₁₋₂, and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl;

R² is selected from the group consisting of H, C₁₋₁₄alkyl, C4-10cycloalkyl, (CH₂)₁₋₄aryl, 1-(3-isopropenylphenyl)-1-methylethyl, 1,3-dioxo-1,3-dihydroisoindol-2-ylmethyl and C₂₋₈alkenyl;

R³ represents a suitable primary amine substituent; and

X is selected from the group consisting of a covalent bond, CONH and CO, with the proviso that when X is a covalent bond, R² is H.

This method comprises the steps of:

treating in an inert solvent, a compound of Formula 1:

SS—OH (1)

with a compound or an array of compounds of Formula 2:

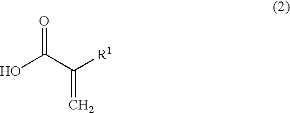

in the presence of a dehydrating agent and an optional catalyst, to form a compound or an array of compounds of Formula 3:

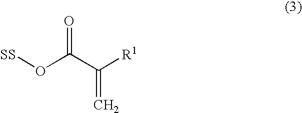

where SS represents a solid support and R¹ is as defined above;

treating an inert solvent mixture of a compound or an array of compounds of Formula 3 with a compound of Formula 4:

PG—CH—N₂ (4)

to form a compound or an array of compounds of Formula 5:

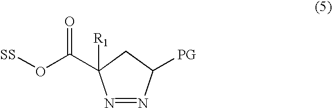

where PG represents a protecting group and SS and R¹ are as defined above;

treating an inert solvent solution of a compound of Formula 5 with a deprotecting agent to form a compound of Formula 6:

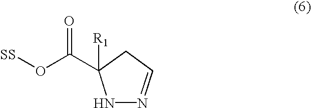

where SS and R¹ are as defined above;

optionally treating an inert solvent mixture of a compound or an array of compounds of Formula 6 with a compound or an array of compounds of Formula 7:

R²—W (7)

to form a compound or an array of compounds of Formula 8:

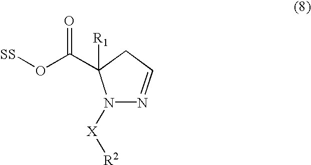

where W represents —COCl, —N═C═O or —COOH; and SS, R¹, R² and X are as defined above; and

treating a solution of a compound or an array of compounds of Formula 6 or Formula 8 with a compound or an array of compounds of Formula 9:

R³—NH₂ (9)

in the presence of an optional base to form a compound of Formula I.

Provided in a preferred embodiment is a method wherein a compound or an array of compounds of Formula 6 is treated with a compound or an array of compounds of Formula 7 to form a compound or an array of compounds of Formula 8.

In another embodiment of the invention, the base in step (i) is a tertiary amine. Preferably the tertiary amine is selected from the group consisting of diisopropylethylamine, TEA, NMM and lutidine.

Another embodiment of the invention utilizes an inert solvent in step (ii) that is selected from the group consisting of toluene, THF, hexanes and CH₂Cl₂, and the compound of Formula 4 in step (ii) is trimethylsilyl diazomethane.

In yet another embodiment, the deprotecting agent in step (iv) is selected from the group consisting of TFA and silver triflate; the compound of Formula 7 in step (iv) is an isocyanate represented by Re—N═C═O; step (iv) utilizes a solution of a compound of Formula 8 in pyridine, THF, dioxane or mixtures thereof; and step (v) is carried out in the presence of a base selected from the group consisting of TEA and DIPEA.

A particularly preferred method of the present invention provides a process wherein:

R¹ is selected from the group consisting of C₁₋₈alkyl; (CH₂)₁₋₂—Ph; 4-methoxybenzyl; 4-methoxyphenylethyl; 3,4-dimethoxybenzyl; 2-benzyloxyethyl; 2-benzyloxypropyl; 3-methoxy-2-benzyloxyethyl; and 3-trifloromethyl-2-benzyloxyethyl;

R² is selected from the group consisting of methyl, ethyl, i-propyl, propyl, t-butyl, cyclohexyl, phenylethyl, benzyl, CH₂—CH═CH₂, 1-(3-isopropenylphenyl)-1-methylethyl and 1,3-dioxo-1,3-dihydroisoindol-2-ylmethyl; and

R³ is selected from the group consisting of 2-hydroxy-1-hydroxymethyl-2-phenylethyl; 2-hydroxy-1-hydroxymethyl-3-methyl-pentyl; 1-benzyl-2-hydroxyethyl; 1-hydroxymethyl-3-methylbutyl; 4-amino-piperidine-1-carboxylic acid ethyl ester; 2-acetylaminoethyl; 2-diethylaminoethyl; 2-(2-hydroxyethoxy)ethyl; 3-diethylaminopropyl; 3-hydroxypropyl; 6-hydroxyhexyl; 3-imidazol-1-yl-propyl; 2-(4-sulfamoylphenyl)-ethyl; 3-(4-methylpiperazin-1-yl)-propyl; 2-dimethylamino-1-methylethyl; 2-[bis-(2-hydroxyethyl)-amino]-ethyl; 1-carbamoyl-2-phenylethyl; 2-dibutylaminoethyl; 5-hydroxy-4,4-dimethylpentyl; 3-dimethylamino-2,2-dimethylpropyl; 2-(butylethylamino)ethyl; 2-diisobutylaminoethyl; 2-hydroxybutylamino; 3-hydroxy-2,2-dimethylpropyl; cyclohexyl; (5-hydroxy-1,3,3-trimethylcyclohexylmethyl); 1,2,3,4-tetrahydronaphthalen-1-yl; cyclooctyl; 3-(2-oxo-pyrrolidin-1-yl)propyl; indan-1-yl; (tetrahydrofuran-2-ylmethyl)amino; 2-(1H-indol-3-yl)ethyl; (benzo[1,3]dioxol-5-ylmethyl); 3-morpholin-4-yl-propyl; 2-pyridin-2-yl-ethyl; 2-hydroxy-1-methyl-2-phenylethyl; 1-methoxyethyl; 1-methyl-3-phenylpropyl; 3-diethyl-1-methylpropyl; benzyl; 2-fluorobenzyl; 2-methoxybenzyl; 3-trifluoromethylbenzyl; 2-phenylaminoethyl; 2-methoxyethyl; phenylethyl; 2-(2-methoxy-phenyl)-ethyl; 2-(3,4-dimethoxyphenyl)ethyl; 2-(4-chlorophenyl)ethyl; 2-(4-methoxyphenyl)ethyl; 2-(4-hydroxyphenyl)ethyl; 3,3-diphenylpropyl; 2,5-dimethylbenzyl; 2-trifluoromethylbenzyl; butyl; 1,2-diethylpyrazolidin-4-yl; 3-methoxypropyl; 2-diisopropylaminoethyl; 1-isopropyl-2-methylpropyl; 3-m-tolylaminopentyl; 3-butoxypropyl; 1-(4-fluorophenyl)ethyl; 1-methoxymethylpropyl; 2,3-dimethoxybenzyl; 2,4-dimethoxybenzyl; 2-(2-chloro-6-fluoro-benzylsulfanyl)ethyl; 2,6-dimethoxybenzyl; 3,5-dimethoxybenzyl; 2-phenoxyethyl; 1-benzylpyrrolidin-3-yl; 2-(2,3-dimethoxy-phenyl)ethyl; 2-(2,5-dimethoxy-phenyl)-ethyl; 2-(2-ethoxyphenyl)-ethyl; 2-(3,5-dimethoxyphenyl)ethyl; 2-(4-ethoxyphenyl)-ethyl; 2-(4-trifluoromethoxyphenyl)ethyl; 2-hydroxy-1,2-diphenylethyl; 2-hydroxy-1,2-diphenylethyl; 2-(2-hydroxymethylphenylsulfanyl)benzyl; 2-(3-fluoro-phenyl)ethyl; 2-(2-aminophenyl)benzyl; 2-(2-fluorophenyl)ethyl; 4-aminobenzyl; 2-(3,4-dimethoxyphenyl)ethyl; 1,2-dihydroxy-2-(4-methylsulfanylphenyl)ethyl; 2-hydroxycyclohexyl; 3-(methylphenylamino)propyl; piperonyl; phenyl-N-methylpropyl; hydroxyethyloxyethyl; 2-indolinyl ethyl; and benzyl; and

X is selected from the group consisting of CONH and CO.

The present invention also provides a method for synthesizing a compound or an array of compounds of Formula 5 comprising treating an inert solvent mixture of a compound or an array of compounds of Formula 3:

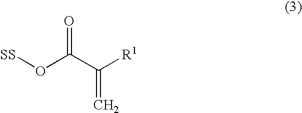

with a compound of Formula 4:

PG—CH—N₂ (4)

to form a compound or an array of compounds of Formula 5:

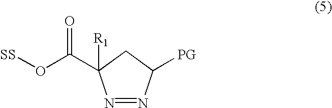

where PG represents a protecting group and SS represents a solid support and R¹ is selected from the group consisting of C₁₋₁₄alkyl, (CH₂)₀₋₆aryl, (CH₂)₀₋₂CH(Ph)₂, (CH₂)₁₋₄aryl(C₁₋₄ alkoxy)₁₋₃, (CH₂)₀₋₄-Z-(CH₂)1-4aryl (C₁₋₄alkoxy)₀₋₄, (CH₂)1₄-Z-aryl-(CF₃)1-2, and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl.

The present invention also provides a method for synthesizing a compound or an array of compounds of Formula 6 comprising treating an inert solvent solution of a compound of Formula 5:

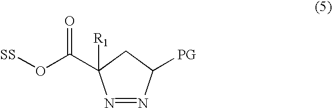

with a deprotecting agent to form a compound of Formula 6:

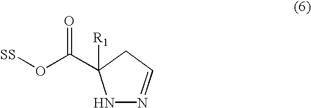

where PG represents a protecting group and SS represents a solid support and R¹ is selected from the group consisting of C₁₋₁₄alkyl, (CH₂)₀₋₆aryl, (CH₂)₀₋₂CH(Ph)₂, (CH₂)₁₋₄aryl(C₁₋₄ alkoxy)₁₋₃, (CH₂)₀₋₄-Z-(CH₂)₁₋₄aryl (Cl₄alkoxy)₀₋₄, (CH₂)₁₋₄-Z-aryl-(CF₃)₁₋₂, and C₂₋₁₄alkenyl substituted with up to three substituents selected from the group consisting of Ph and C₁₋₁₄alkyl, where Z is selected from the group consisting of O, S and NR⁴ where R⁴ is C₁₋₁₄alkyl or aryl.

The present invention also provides a method for synthesizing a compound or an array of compounds of Formula 8 treating an inert solvent mixture of a compound or an array of compounds of Formula 6:

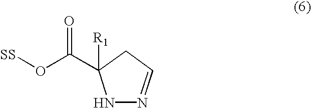

with a compound or an array of compounds of Formula 7:

R—W (7)

where W represents —COCl, —N═C═O or COOH, to form a compound or an array of compounds of Formula 8:

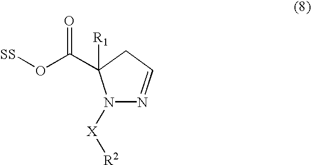

wherein:

PG represents a protecting group;

SS represents a solid support;

R¹ is selected from the group consisting of C₁₋₁₄alkyl; (CH₂)₀₋₆aryl; (CH₂)₀₋₂CH(Ph)₂; (CH₂)₁₋₄aryl(C₁₋₄alkoxy)₁₋₃; (CH₂)₀₋₄-Z-(CH₂)₁₄aryl(C₁₋₄alkoxy)₀₋₄; (CH₂)₁₋₄-Z-aryl-(CF₃)_{1-2;} and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl;

R² is selected from the group consisting of H, C₁₋₁₄ straight chain or branched alkyl; C₄₋₁₀cycloalkyl; (CH₂)₁₋₄aryl; 1-(3-isopropenylphenyl)-1-methylethyl; phenylethyl; 1,3-dioxo-1,3-dihydroisoindol-2-ylmethyl and C₂₋₈alkenyl; and

X is selected from the group consisting of a covalent bond, CONH and CO, with the proviso that when X is a covalent bond, R² is H.

Another aspect of the present invention provides novel compounds of Formula 5, Formula 6 and Formula 8. These compounds are useful intermediates in the synthesis of pyrazoline based compounds.

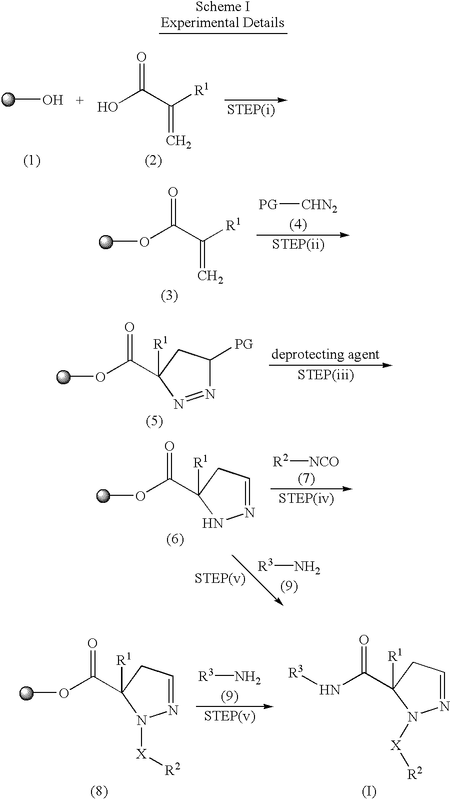

Synthetic Procedure:

STEP (i)

A mixture of a compound of Formula 2 (1 eq.) and a dehydrating agent (e.g., DIC; 1 eq.) in an inert solvent (e.g., methylene chloride, THF) is agitated for about 5-30 minutes. The mixture then is added to a methylene chloride solution of a compound of Formula 1 to form a slurry. The slurry then is combined with a base (e.g., NMM, 0.5 to 1.5 q.) and a catalyst (e.g., DMAP, 0.05 to 0.15 eq.) and the resulting mixture is stirred for about 12-24 hours to form a compound of Formula 3, generally as a solid. The solid is isolated and washed four times in succession with CH₂Cl₂, DMF, CH₃OH, CH₂Cl₂ and ether to yield a compound of Formula 3.

STEP (ii):

A compound of Formula 3 is placed in a round bottomed flask. An inert solvent, preferably toluene, is added to form a slurry. This slurry is diluted with about 4-6 equivalents of a compound of Formula 4, preferably trimethylsilyl diazomethane. The diluted slurry is agitated for about 12-24 hours at ambient temperature to form a compound of Formula 5, in the form of a resin. This resin is isolated, washed (CH₂Cl₂x3) and dried to yield a compound of Formula 5.

STEP (iii):

A mixture of methylene chloride and a compound of Formula 5 (1 eq.) is mixed with about 2-18 equivalents of a deprotecting agent, (e.g., TFA) to form a slurry. The slurry is let stand at ambient temperature for about 1-4 hours to form a suspension. The suspension is filtered yielding a solid which is washed in succession with CH₂Cl₂, a mixture of 10% NMM/CH₂Cl₂, CH₃OH, CH₂Cl₂ and diethyl ether. This washing sequence is repeated for up to about three times and the washed solid is dried to yield a compound of Formula 6.

STEP (iv)

A compound of Formula 6 (1 eq.) in an inert solvent (e.g., CH₂Cl₂, THF) is combined with a compound of Formula 7 to form a mixture. Preferred compounds of Formula 7 are isocyanates, however carboxylic acids and acid chlorides can also be used. The mixture is agitated for about 8-200 hours, or until the compound of Formula 6 is essentially completely consumed, to form solids. The level of consumption of the compound of Formula 6 can be checked by LC/MS, TLC or other methods known to one skilled in the art. The solids are isolated and washed in succession with CH₂Cl₂ and ether. This washing sequence is repeated 3-4 times and the washed solid is dried to yield a compound of Formula 8.

STEP (v)

A compound of Formula 6 and/or Formula 8 are independently mixed with a solution of an amine of Formula 9. Note than use of a compound of Formula 6 in this step will provide a compound of Formula I where X is a covalent bond and R² is H. It is preferable to use the amine of Formula 9 in the form of a solution in a basic solvent. Illustrative examples of basic solvents are TEA and pyridine. The preceding mixture is agitated for about 12-72 hours. The mixture is filtered and the residue is washed with pyridine (x2). The combined filtrate is concentrated under reduced pressure to yield a compound of Formula I. Compounds of Formula I can be purified by methods known to one skilled in the art.

### Synthesis of an Array of Compounds of Formula I

A mixture of a compound of Formula 6 or Formula 8 (1 eq.) and an inert solvent is placed in each well of a Polyfiltronics plate. The plate is sealed from the bottom and then is placed in a clamp. A structurally independent and distinct amine of Formula 9 (1-3 eq.) then is added to the contents of each well. It is preferable to use the amine of Formula 9 in the form of a solution in a basic solvent. Illustrative examples of basic solvents are TEA and pyridine. The Polyfiltronics plate then is sealed from the top and the well contents are agitated at ambient temperature for about 6-72 hours. The liquid contents of each well are drained from the bottom. The reaction solids then are washed with pyridine (×2). The combined filtrates from individual wells are independently concentrated under reduced pressure to yield an array of compounds of Formula I.

### Synthesis of Starting Materials

Compounds of Formula 1

Compounds of Formula 1 are commercially available from Aldrich Chemicals.

Compounds of Formula 2

Compounds of Formula 2 are commercially available from Aldrich Chemicals. The following representative procedures can also be used to synthesize compounds of Formula 2.

### Synthesis of 2-Benzylacrylic acid (a compound of Formula 2)

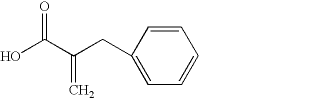

A mixture of 3-phenylpropionaldehyde (89.0 g, 0.663 mol, 1 eq., Aldrich), CH₂Cl₂ (2 L, HPLC grade) and TEA (550 mL, 3.95 mol, 6 eq., Aldrich) was agitated at about 10° C. for about 30 minutes. Eschenmoser's salt (151 g, 0.817 mol, 1.25 eq., Aldrich) was then added in one portion, and the reaction mixture was agitated for about 12-18 hours at ambient temperature. The reaction mixture then was washed in succession with saturated aqueous NaHCO₃ (500 mL) and saturated aqueous NaCl (500 mL), the organic layers were dried (MgSO₄) and concentrated under reduced pressure to yield a dark yellow oil. The yellow oil was diluted with diethyl ether, filtered through silica and the filtrate is concentrated to yield 99.3 g of 2-exomethylene aldehyde as a yellow oil.

The above 2-exomethylene aldehyde (0.612 mol, 1 eq.) was dissolved in a mixture of t-butanol (765 mL) and 2-methyl-2-butene (300 mL) and this reaction mixture was agitated at ambient temperature for about 30 minutes. This reaction mixture then was diluted with an aqueous mixture of about 80% sodium chlorite (69.2 g, 0.612 mol, 1 eq.) and NaH₂PO₄ (84.5 g, 0.612 mol, 1 eq.) in water (600 mL) over a period of about 1.5 hours. The resulting reaction mixture was agitated at ambient temperature for up to about 24 hours and concentrated under reduced pressure to yield a residue. The residue was diluted with water (2 L) and washed with hexanes (2×250 mL). The aqueous layer was acidified to pH 3 with 1 M HCl, extracted with CH₂Cl₂ (3×1000 mL), the combined organic layers were dried (MgSO₄), filtered and concentrated under reduced pressure to yield 49.2 g (0.304 mol, 46%) of 2-benzylacrylic acid as a white solid.

Molecular Formula: C₁₀H₁₀O₂; Molecular Weight: 162.19

¹H NMR (270 MHz, CDCl₃) δ: 10.28 (s, 1 H), 7.52-7.03 (m, 5 H), 6.39 (s, 1 H), 5.58 (d, J=1.2 Hz, 1 H), 3.69 (s, 2 H).

¹³C NMR (67.5 MHz, CDCl₃) δ: 172.2, 129.2, 129.1. 128.8, 128.6, 126.7, 126.5, 37.7

MS (CI) m/z 163 [(M+H)⁺].

Synthesis of 2-(4-Methoxybenzyl)acrylic acid (a compound of Formula 2)

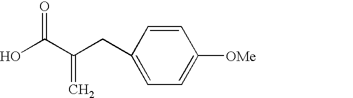

A mixture of 3-(4-methoxyphenyl)-1-propanol (74.5 g, 0.438 mol, 1 eq., Aldrich) in DMSO (1 L, HPLC grade), TEA (360 mL, 2.63 mol, 6 eq.,) and a sulfur trioxide-pyridine complex (78.2 g, 0.482 mol, 1.1 eq.) in DMSO (0.5 L, HPLC grade) was agitated for about 30 minutes in a nitrogen atmosphere. Eschenmoser's salt (152 g, 0.823 mol, 1.9 eq., Aldrich) then was added in one portion and the resulting reaction mixture was stirred for about 12-18 hours.

This reaction mixture was diluted with cold saturated aqueous NaHCO₃ (750 mL) and diethyl ether (500 mL). The organic layer was separated and washed with saturated aqueous NaCl (250 mL), dried (MgSO₄), filtered and concentrated under reduced pressure to yield a dark yellow oil. This yellow oil was diluted with diethyl ether, filtered through silica and concentrated to yield 70.0 g of 2-exomethylene aldehyde.

The above 2-exomethylene aldehyde (0.360 mol, 1 eq.) was dissolved in a mixture of t-butanol (450 mL) and 2-methyl-2-butene (200 mL) and the mixture agitated at a temperature below about 30° C. An aqueous mixture of sodium chlorite (40.7 g, 0.360 mol) and NaH₂PO₄ (49.7 g, 0.360 mol) in water (360 mnL) was added in portions, to the agitated reaction mixture over a period of 1.5 hours. The resulting reaction mixture was stirred for about 15-24 hours at ambient temperature and then concentrated under reduced pressure to yield a residue. The residue was diluted with water (750 mL) and washed with hexanes (2×150 mL). The aqueous layer was acidified to pH 2 with 1 M HCl (ca. 220 mL) and extracted with CH₂Cl₂ (3×500 mL). The combined organic layers are dried (MgSO₄) and concentrated under reduced pressure to provide 31.1 g (0.162 mol.) of 2-(4-methoxybenzyl)acrylic acid as a pale yellow solid.

Molecular Formula: C₁₁H₁₂O₃; Molecular Weight: 192.21

¹H NMR (270 MHz, CDCl₃) δ: 7.11 (d, J=8.4 Hz, 2 H), 6.83 (d, J=8.4 Hz, 2 H), 6.32 (s, 1 H), 5.53 (s, 1 H), 3.77 (s, 3 H), 3.55 (s, 2 H).

¹³C NMR (67.5 MHz, CDCl₃) δ: 171.8, 158.1, 140.0, 130.5, 130.0, 129.3, 128.0, 55.2, 36.7.

MS (ESI) m/z 193 [(M+H)⁺].

Synthesis of 2-(2-Benzyloxyethyl)acrylic acid (a compound of Formula 2)

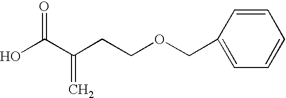

A suspension of 60% sodium hydride (23.4 g, 0.585 mol, 1 eq.) in anhydrous THF (2 L, HPLC grade) was agitated at about 0° C. The suspension was diluted with 1,4-butanediol (260 mL, 2.03 mol, 3.5 eq., Fluka) and the resulting mixture was agitated at a temperature of about 0° C. for about an hour. The mixture then was diluted with benzyl bromide (100 g, 0.585 mol, 1 eq.) and this mixture is stirred at ambient temperature for about 12-24 hours. The reaction mixture was quenched with piperidine (10 mL) and this quenched mixture was agitated for about one hour. The quenched reaction mixture was extracted with aqueous NH₄Cl solution (100 mL), the aqueous layer then was extracted with CH₂Cl₂ (×3). The organic layers were combined, dried (MgSO₄) and concentrated under reduced pressure to yield an oily reside. The oily residue comprised 1,4-butanol which was removed by distillation and the remaining residue was diluted with a 1:1 mixture of hexanes:ethyl acetate. The diluted mixture was filtered through silica and the filtrate concentrated to yield 4-benzyloxy-butan-1-ol (102 g).

A mixture of 4-benzyloxy-butan-1-ol (53 g; 0.294 mol; 1 eq.) and TEA (245 mL. 1.76 mol., 6 eq.) in DMSO was mixed with sulfur trioxide-pyridine complex (93.7 g; 0.589 mol; 2 eq.) in DMSO (0.3 L, HPLC grade) over a period of about 15 minutes. This mixture was agitated for about 1.5 hours and then dimethylmethyleneiminium chloride 934. g; 0.368 mol; 1.25 eq.) was added in one portion. The resulting reaction mixture was agitated for about 12 to about 24 hours, mixed with saturated aqueous NaHCO₃ (1500 mL) and extracted with ether (1000 mL). The organic layer was isolated, washed in succession with water (1000 mL) and NaCl (1000 mL), dried (MgSO₄) and concentrated under reduced pressure to yield an oily residue. This residue was diluted with ether, filtered through silica and concentrated under reduced pressure to yield 2-oxomethylene aldehyde (66.1 g).

A mixture of 66.1 g of the above 2-exomethylene aldehyde (0.294 mol), t-butanol (435 mL) and 2-methyl-2-butene (174 mL) was agitated at ambient temperature. The agitated mixture was combined with a mixture of 80% sodium chlorite (39.3 g, 0.348 mol) and NaH₂PO₄ (48.0 g, 0.348 mol, 1.5 eq.) in water (348 mL) over a period of 1.5 hours. The resulting mixture was stirred for 18 hours at ambient temperature, and concentrated under reduced pressure to remove the t-butanol leaving behind an aqueous residue. The aqueous residue was diluted with water (1500 mL) and washed with hexanes (2x 300 mL). The aqueous layer was acidified to pH 3 with 1 M HCl (250 mL) and extracted with CH₂Cl₂ (3×500 mL). The combined organic layers were dried (MgSO₄) and concentrated under reduced pressure to yield 38.3 g (0.186 mol, 53%, overall) of the title compound.

Molecular Formula: C₁₂H₁₄O₃; Molecular Weight: 206.24

¹H NMR (270 MHz, CDCl₃) δ: 7.39-7.24 (m, 5 H), 3.65 (s, 1 H), 5.75 (d, J=1.0 Hz, 1 H), 4.52 (s, 3 H), 3.65-3.60 (m, 2 H), 2.65-2.60 (m, 2 H).

¹³C NMR (67.5 MHz, CDCl₃)δ: 171.7, 138.1, 136.9, 128.8, 128.3, 127.7, 127.6, 72.8, 68.6, 31.9.

MS (ESI) m/z 207 [(M+H)⁺].

Synthesis of 2-phenylethyl-acrylic acid (a compound of Formula 2)

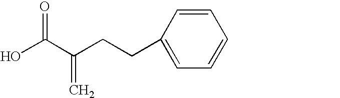

In a 5 L round-bottomed flask, 4-phenyl-1-butanol (99.2 g, 0.660 mol, Aldrich) was dissolved in DMSO (3.4 L) and TEA (552 mL, 3.96 mol, 6 equiv, Aldrich,) under an atmosphere of nitrogen. With stirring at ambient temperature, sulfur trioxide-pyridine complex (210 g, 1.32 mol, 2.0 equiv, Aldrich) was added as a solid over 20 minutes. After stirring for 1.5 hours, dimethylmethyleneiminium chloride (77.1 g, 0.830 mol, 1.25 equiv, Fluka) was added in one portion. After stirring overnight (about 14 hours), the reaction mixture was poured into a stirring biphasic mixture of cold saturated aqueous NaHCO₃ (1.65 L) and diethylether (1.10 L).

The organic layer was separated from the aqueous phase, and washed with water (800 mL), then saturated aqueous NaCl (800 mL). The organic layer was concentrated to a minimum volume in vacuo. The crude material was then passed through a silica plug with diethylether until product completely eluted out, and concentrated to 116 g of 2-phenethyl-propenal containing some residual DMSO. The material was carried immediately on to the next step.

In a 3 L round-bottomed flask, 2-phenethyl-propenal (105.7 g, 0.660 mol) was dissolved in tert-butanol (975 mL, Aldrich) and 2-methyl-2-butene (390 mL, Aldrich). Stirring the mixture and maintaining the internal temperature below 25° C. with an ice-water bath, a solution of 80% sodium chlorite (88.0 g, 0.790 mol, Acros) and NaH₂PO₄ (107.5 g, 0.790 mol, Acros) in water (780 mL) was added dropwise over 1.5 hours. After vigorous stirring for 18 hours at ambient temperature, the reaction mixture was distilled in vacuo to remove all tert-butanol. The residue was diluted with water (1.50 L) and washed with hexanes (2×600 mL). The desired product was in the organic layer, and the product and an undesired side-product was in the aqueous layer. Additional extractions with hexanes (7×600 mL) removed extracted product out of the aqueous layer. The aqueous layer was then extracted with 1:1 hexanes/diethylether (600 mL), and this organic layer was washed with saturated aqueous NaHCO₃. The combined organic layers were then concentrated in vacuo to provide 54.5 g (0.309 mol, 47% overall yield) of 2-phenylethyl-acrylic acid as a yellow oil.

¹H NMR (270 MHz, CDCl₃) δ 10.50 (s, 1H), 7.31 (m, 2H), 7.22 (m, 3H), 6.33 (s, 1H), 5.62 (s, 1H, 2.85 (t, J=8.4 Hz, 2H), 2.65 (t, J=8.6 Hz, 2H)

¹³CNMR(67.5 MHz, CDCl₃) δ 172.2, 141.2, 139.4, 128.4, 128.3, 127.5, 125.9, 34.7, 33.4

MS (EI) m/z 174.2 [(M−H)⁻].

Synthesis of 2-[2-(4-methoxy-phenyl)-ethyl]-acrylic acid (a compound of Formula 2)

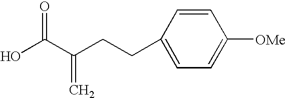

In a 5 L round-bottomed flask equipped with a overhead stir bar, 4-(4-methoxyphenyl)-1-butanol (120 g, 0.660 mol, Aldrich) was dissolved in DMSO (3.4 L) and TEA (552 mL, 3.96 mol, 6 equiv, Aldrich,) under an atmosphere of nitrogen. With stirring at ambient temperature, sulfur trioxide-pyridine complex (210 g, 1.32 mol, 2.0 equiv, Aldrich) was added as a solid over 20 minutes. After stirring for 1.5 hours, dimethylmethyleneiminium chloride (77.1 g, 0.830 mol, 1.25 equiv, Fluka) was added in one portion. After stirring overnight (about 11 hours), the reaction mixture was poured into a stirring biphasic mixture of cold saturated aqueous NaHCO₃ (1.60 L) and diethylether (1.10 L). After bubbling subsided (about 10 minutes), the organic layer was separated from the aqueous phase. The aqueous phase was extracted with diethyl ether (1×400 mL). The combined organic layers were washed with water (800 mL), then saturated aqueous NaCl (800 mL). The organic layer was concentrated to a minimum volume in vacuo. The crude material was then passed through a silica plug with diethylether until product completely eluted out, and concentrated to 139 g of 2-[2-(4-methoxy-phenyl)-ethyl]-propenal containing some residual DMSO. The material was carried immediately on to the next step.

In a 3 L round-bottomed flask, 2-[2-(4-methoxy-phenyl)-ethyl]-propenal (0.660 mol) was dissolved in tert-butanol (975 mL, Aldrich) and 2-methyl-2-butene (390 mL, Aldrich). Gently stirring the mixture and maintaining the internal temperature below 20° C. with an ice-water bath, a solution of 80% sodium chlorite (88.0 g, 0.790 mol, Acros) and NaH₂PO₄ (107.5 g, 0.790 mol, Acros) in water (780 mL) was added dropwise over 2 hours. After gently stirring for 18 hours at ambient temperature, the reaction mixture was distilled in vacuo to remove tert-butanol. The residue was diluted with water (1.50 L) and washed with hexanes (2×200 mL). Some of the desired product was in the organic layer, but most of the product was in the aqueous layer. The aqueous layer was acidfied from pH 7 to pH 3 with 50% H₃PO₄/H₂0 (v/v) and extracted with CH₂Cl₂ (3×400 mL). The combined organic layers were then concentrated in vacuo to provide 97.5 g (0.473 mol, 72% overall yield) of 2-[2-(4-methoxy-phenyl)-ethyl]-acrylic acid as a light yellow solid.

¹H NMR (270 MHz, CDCl₃) δ 7.09 (d, J=8.6 Hz, 2H), 6.81 (d, J=8.91 Hz, 2H), 6.28 (s, 1H), 5.58 (s, 1H), 3.77 (s, 3H), 2.73 (t, J=8.6 Hz, 2H), 2.58 (t, J=8.2 Hz, 2H)

¹³C NMR (67.5 MHz, CDCl₃) δ 172.2, 157.9, 139.2, 133.3, 129.4, 127.8, 113.7, 55.2, 33.88, 33.76

MS (El) m/z 204.7 [(M−H)⁻]

Synthesis of 2-(3-benzyloxy-propyl)-acrylic acid (a compound of Formula 2)

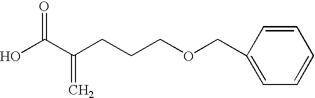

In a 5 L round-bottomed flask, 5-benzyloxypentanol (128 g, 0.660 mol, Aldrich) was dissolved in DMSO (2.8 L) and TEA (551 mL, 3.96 mol, 6 equiv, Aldrich,) under an atmosphere of nitrogen. With stirring at ambient temperature, sulfur trioxide-pyridine complex (210 g, 1.32 mol, 2.0 equiv, Aldrich) was added as a solid over 20 minutes. After stirring for 1.5 hours, dimethylmethyleneiminium chloride (77.1 g, 0.830 mol, 1.25 equiv, Fluka) was added in one portion. After stirring overnight (about 11 hours), the reaction mixture was poured into a stirring biphasic mixture of cold saturated aqueous NaHCO₃ (1.80 L) and diethylether (1.30 L). After bubbling subsides (about 10 minutes), the organic layer was separated from the aqueous phase. The aqueous phase was extracted with diethyl ether (1×1.40 L). The combined organic layers were washed with water (800 mL), then saturated aqueous NaCl (800 mL). The organic layer was concentrated to a minimum volume in vacuo. The crude material was then passed through a silica plug with diethylether until product completely eluted out, and concentrated to 140 g of 2-(3-benzyloxy-propyl)-propenal as a light yellow viscous oil containing some residual DMSO. The material was carried immediately on to the next step.

In a 3 L round-bottomed flask, 2-(3-benzyloxy-propyl)-propenal (0.660 mol) was dissolved in tert-butanol (976 mL, Aldrich) and 2-methyl-2-butene (392 mL, Aldrich). Gently stirring the mixture and maintaining the internal temperature below 25° C. with an ice-water bath, a solution of 80% sodium chlorite (88.0 g, 0.790 mol, Acros) and NaH₂PO₄ (107.5 g, 0.790 mol, Acros) in water (780 mL) was added dropwise over 2 h. After gently stirring for 40 h at ambient temperature, the reaction mixture was distilled in vacuo to remove tert-butanol. The residue was diluted with water (1.50 L) and washed with hexanes (1×200 mL). The product remained in the aqueous layer. The aqueous layer was extracted with CH₂Cl₂ (3×400 mL). The combined organic layers were then dried over anhydrous Na₂SO₄, filtered, and concentrated in vacuo to provide 108 g (0.490 mol, 74% overall yield) of 2-(3-benzyloxy-propyl)-acrylic acid as a yellow solid.

¹H NMR (270 MHz, CDCl₃) δ 9.23 (s, 1H), 7.33 (m, 5H), 6.25 (s, 1H), 5.60 (s, 1H, 4.49 (s, 2H), 3.49 (t, J 6.3 Hz, 2H), 2.40 (t, J 7.3 Hz, 2H), 1.81 (q, J 7.6 Hz, 2H)

¹³C NMR (67.5 MHz, CDCl₃) δ 171.3, 139.7, 138.3, 128.2, 127.6, 127.4, 126.5, 72.7, 69.3, 28.2

MS (EI) m/z 219.2 [(M−H)⁻].

Synthesis of 2-(3,4-dimethoxy-benzyl)-acrylic acid (a compound of Formula 2)

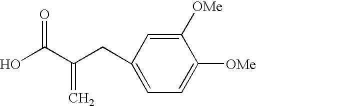

In a 5 L round-bottomed flask, 3-(3,4-dimethoxy phenyl)-1-propanol (130 g, 0.660 mol, Aldrich) was dissolved in DMSO (2.8 L) and TEA (551 mL, 3.96 mol, 6 equiv, Aldrich,) under an atmosphere of nitrogen. With stirring at ambient temperature, sulfur trioxide-pyridine complex (210 g, 1.32 mol, 2.0 equiv, Aldrich) was added as a solid over 20 minutes. After stirring for 1.5 hours, dimethylmethyleneiminium chloride (77.1 g, 0.830 mol, 1.25 equiv, Fluka) was added in one portion. After stirring overnight (about 11 hours), the reaction mixture was poured into a stirring biphasic mixture of cold saturated aqueous NaHCO₃ (1.80 L) and diethylether (1.10 L). The aqueous phase was extracted with hexane (3×400 mL). The organic layers were washed with water (400 mL), then saturated aqueous NaCl (800 mL). The organic layer was concentrated to a minimum volume in vacuo. The crude material was then passed through a silica plug with diethylether until product completely elutes out, and concentrated to 150 g of 2-(3,4-dimethoxy-benzyl)-propenal as a light yellow viscous oil containing some residual DMSO. The material was carried immediately on to the next step.

In a 3 L round-bottomed flask, 2-(3,4-dimethoxy-benzyl)-propenal (0.660 mol) was dissolved in tert-butanol (976 mL, Aldrich) and 2-methyl-2-butene (392 mL, Aldrich). Gently stirring the mixture and maintaining the internal temperature below 25° C. with an ice-water bath, a solution of 80% sodium chlorite (88.0 g, 0.790 mol, Acros) and NaH₂PO₄ (107.7 g, 0.790 mol, Acros) in water (780 mL) was added dropwise over 1.5 hours. After gently stirring for 18 hours at ambient temperature, the reaction mixture was distilled in vacuo to remove tert-butanol. The residue was diluted with water (1.50 L) and washed with hexanes (1×200 mL). The product remained in the aqueous layer. The aqueous layer was acidfied from pH 7 to pH 3 with 1 N HCl and extracted with CH₂Cl₂ (3×600 mL). The combined organic layers were then concentrated in vacuo to provide 88.6 g (0.399 mol, 60% overall yield) of 2-(3,4-dimethoxy-benzyl)-acrylic acid as a viscous yellow oil.

¹H NMR (270 MHz, CDCl₃) δ 10.30 (s, 1H), 6.76-6.10 (m, 3H), 6.24 (s, 1H), 5.45 (s, 1H), 3.77 (s, 6H), 3.50 (s, 2H)

¹³C NMR (67.5MHz, CDCl₃) δ 171.0, 148.7, 147.4, 140.0, 131.0, 127.4, 120.9, 112.2, 111.1, 55.7, 55.6, 39.9

MS (EI) m/z 221.1 [(M−H)⁻].

Synthesis of 2-[2-(3-methoxy-benzyloxy)-ethyl]-acrylic acid (a compound of Formula 2)

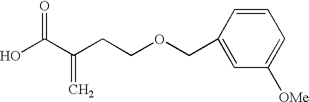

In a 4-neck 5 L round-bottomed flask, 60% sodium hydride (40 g, 1.0 mol, 1 equiv, Aldrich) was suspended in anhydrous THF (2 L, HPLC grade) and cooled to 0° C. 1,4-Butanediol (444 mL, 5.0 mol, 5 equiv, Fluka) was added dropwise over 30 minutes. After stirring at 0° C. for 1 hour, 3-methoxy benzyl bromide (140 mL, 1.0 mol, 1 equiv, Aldrich) was added in one portion. The reaction mixture was allowed to warm to ambient temperature overnight. After 15 hours, piperidine (17 mL) was added to quench any remaining 3-methoxy benzyl bromide and allowed to stir for 1 hour. The reaction mixture was diluted with saturated aqueous NH₄Cl (170 mL), and the filtered through a sintered glass funnel. After diluting the filtrate with water (500 mL), the layers were separated. The aqueous layer was extracted with CH₂Cl₂ (3×500 mL), the combined organic layers dried over anhydrous MgSO₄ for 30 min, filtered, and concentrated in vacuo to a yellow viscous oil. The crude material was then passed through a silica plug with 1:1 hexanes/ethyl acetate until product completely elutes out, and concentrated to 204 g (0.970 mol, 97%) of 4-(3-Methoxy-benzyloxy)-butan-1-ol as a clear, colorless oil. The material was carried immediately on to the next step.

In a 5 L round-bottomed flask, 4-(3-methoxy-benzyloxy)-butan-1-ol (139 g, 0.660 mol) was dissolved in DMSO (3.4 L) and TEA (552 mL, 3.96 mol, 6 equiv, Aldrich,) under an atmosphere of nitrogen. With stirring at ambient temperature, sulfur trioxide-pyridine complex (210 g, 1.32 mol, 2.0 equiv, Aldrich) was added as a solid over 20 minutes. After stirring for 1.5 hours, dimethylmethyleneiminium chloride (77.1 g, 0.830 mol, 1.25 equiv, Fluka) was added in one portion. After stirring overnight (about 11 hours), the reaction mixture was poured into a stirring biphasic mixture of cold saturated aqueous NaHCO₃ (1.60 L) and diethylether (1.10 L).

After bubbling subsided (about 10 minutes), the organic layer was separated from the aqueous phase. The organic layer was washed with water (800 mL), then saturated aqueous NaCl (800 mL). The organic layer was concentrated to a minimum volume in vacuo. The crude material was then passed through a silica plug with diethylether until product completely elutes out, and concentrated to 88.7 g of 2-[2-(3-methoxy-benzyloxy)-ethyl]-propenal containing some residual DMSO. The material was carried immediately on to the next step.

In a 3 L round-bottomed flask, 2-[2-(3-methoxy-benzyloxy)-ethyl]-propenal (0.403 mol) was dissolved in tert-butanol (590 mL, Aldrich) and 2-methyl-2-butene (234 mL, Aldrich). Gently stirring the mixture and maintaining the internal temperature below 20° C. with an ice-water bath, a solution of 80% sodium chlorite (43.4 g, 0.480 mol, Acros) and NaH₂PO₄ (64.5 g, 0.480 mol, Acros) in water (468 mL) was added dropwise over 2 hours. After gently stirring for 18 hours at ambient temperature, the reaction mixture was distilled in vacuo to remove tert-butanol. The residue was diluted with water (1.00 L) and washed with hexanes (1×200 mL). Some of the desired product was in the organic layer, but most of the product was in the aqueous layer. The aqueous layer was acidfied from pH 7 to pH 3 with 50% H₃PO4/H₂O (v/v) and extracted with CH₂Cl₂ (3×400 mL). The combined organic layers were washed with 2 N aqueous citric acid (250 mL) and saturated aqueous NaCl (250 mL) to remove baseline impurity. The combined organic layers were then dried over anhydrous Na₂SO₄, filtered, and concentrated in vacuo to provide 65.0 g (0.275 mol, 42% overall yield) of 2-[2-(3-methoxy-benzyloxy)-ethyl]-acrylic acid as a yellow oil.

¹H NMR (270 MHz, CDCl₃) δ 7.24 (t, J=8.0 Hz, 2H), 6.82 (m, 3H), 6.35 (s, 1H), 5.75 (s, 1H), 4.50 (s, 2H), 3.79 (s, 3H), 3.64 (t, J=6.5 Hz, 2H), 2.62 (t, J=7.5 Hz, 2H)

¹³C NMR (67.5 MHz, CDCl₃) δ 171.2, 159.5, 139.6, 136.8, 129.2, 128.4, 119.7, 113.1, 112.6, 72.7, 68.5, 54.9, 31.8

MS (El) m/z 234.8 [(M−H)⁻]

Synthesis of 2-[2-(3-trifluoromethyl-benzyloxy)-ethyl]-acrylic acid (a compound of Formula 2)

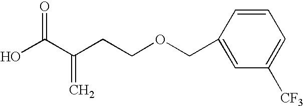

In a 4-neck 5 L round-bottomed flask, 60% sodium hydride (40 g, 1.0 mol, 1 equiv, Aldrich) was suspended in anhydrous THF (2 L, HPLC grade), and cooled to 0° C. 1,4-Butanediol (444 mL, 5.4 mol, 5 equiv, Fluka) was added dropwise over 30 minutes. After stirring at 0° C. for 1 hour, 3-trifluoromethyl benzyl bromide (144 mL, 0.930 mol, 1 equiv, Aldrich) was added in one portion. The reaction mixture was allowed to warm to ambient temperature overnight. After 15 hours, the reaction mixture was diluted with saturated aqueous NH₄Cl (250 mL), and the filtered through a sintered glass funnel. After diluting the filtrate with water (500 mL), the layers were separated. The aqueous layer was extracted with CH₂Cl₂ (3×500 mL), the combined organic layers dried over anhydrous MgSO₄ for 30 minutes, filtered, and concentrated in vacuo to a colorless viscous oil. The crude material was then passed through a silica plug with 1:1 hexanes/ethyl acetate until product completely elutes out, and concentrated to 170 g (0.685 mol, 74%) of 4-(3-trifluoromethyl-benzyloxy)-butan-I-ol as a clear, colorless oil. The material was carried immediately on to the next step.

In a 5 L round-bottomed flask, 4-(3-trifluoromethyl-benzyloxy)-butan-1-ol (164 g, 0.660 mol) was dissolved in DMSO (3.4 L) and triethylamine (552 mL, 3.96 mol, 6 equiv, Aldrich,) under an atmosphere of nitrogen. With stirring at ambient temperature, sulfur trioxide pyridine complex (210 g, 1.32 mol, 2.0 equiv, Aldrich) was added as a solid over 20 minutes. After stirring for 1.5 h, dimethylmethyleneiminium chloride (77.1 g, 0.830 mol, 1.25 equiv, Fluka) was added in one portion. After stirring overnight (about 16 hours), the reaction mixture was poured into a stirring biphasic mixture of cold saturated aqueous NaHCO₃ (1.80 L) and diethylether (1.10 L). After stirring in ice-water bath for 30 min, the organic layer was separated from the aqueous phase. The organic layer was washed with water (800 mL), then saturated aqueous NaCl (800 mL). The organic layer was concentrated to a minimum volume in vacuo. The crude material was then passed through a silica plug with diethylether until product completely elutes out, and concentrated to 160 g of 2-[2-(3-trifluoromethyl-benzyloxy)-ethyl]-propenal containing some residual DMSO. The material was carried immediately on to the next step.

In a 3 L round-bottomed flask equipped with a magnetic stir 2-[2-(3-trifluoromethyl-benzyloxy)-ethyl]-propenal (0.660 mol) was dissolved in tert-butanol (976 mL, Aldrich) and 2-methyl-2-butene (392 mL, Aldrich). Gently stirring the mixture and maintaining the internal temperature below 25° C. with an ice-water bath, a solution of 80% sodium chlorite (88.0 g, 0.781 mol, Acros) and NaH₂PO₄ (108 g, 0.781 mol, Acros) in water (738 mL) was added dropwise over 1.2 hours. After gently stirring for 18 hours at ambient temperature, the reaction mixture was distilled in vacuo to remove tert-butanol. The residue was diluted with water (1.50 L) and washed with hexanes (1×200 mL). Three layers formed. The bottom aqueous layer and the middle oily layer containing most of the desired product were combined, and acidfied from pH 7 to pH 3 with 50% H₃PO₄/H₂O (v/v), and extracted with CH₂Cl₂ (2×500 mL). The combined organic layers, yellow colored due to the presence of residual chloride dioxide, were concentrated to provide 80.0 g (0.292 mol, 44% overall yield) of 2-[2-(3-trifluoromethyl-benzyloxy)-ethyl]-acrylic acid (title compound) as a light yellow oil with 4-(3-trifluoromethyl-benzyloxy)-butan-1-ol as a minor component.

¹H NMR (270 MHz, CDCl₃) δ 7.53 (s, 1H), 7.47-7.34 (m, 3H), 6.27 (d, J=1.2 Hz, 1H), 5.66 (d, J=1.5 Hz, 1H), 4.49 (s, 2H), 3.60 (t, J=6.7 Hz, 2H), 2.60 (t, J=6.4 Hz, 2H)

MS (EI) m/z 273.1 [(M−H)⁻]

Compounds of Formula 4 (PG-CHN₂)

Compounds of Formula 4 are commercially available from Aldrich chemicals, Lancaster and Fluka Chemicals. Compounds typically comprise protecting groups such as trimethyl silyl, triethyl silyl, and t-butyl dimethyl silyl.

Compounds of Formula 7 (R²-W)

Compounds of Formula 7 are available from commercial sources like Aldrich Chemicals, Lancaster and Fluka Chemicals.

Compounds of Formula 9 (R³—NH₂)

Compounds of Formula 9 are primary amines substituted with R³. The method of the present invention essentially can use any substituted primary amine. These substituted primary amines are available from commercial sources such as Aldrich Chemicals, Lancaster and Fluka Chemicals. Illustrative examples of compounds of Formula 9 are: 2-hydroxy-1-hydroxymethyl-2-phenylethylamine; 2-hydroxy-1-hydroxymethyl-3-methylpentylamine; 1-benzyl-2-hydroxyethylamine; 1-hydroxymethyl-3-methylbutylamine; 4-aminopiperidine-1-carboxylic acid ethyl ester; 2-acetylaminoethylamine; 2-diethylaminoethylamine; 2-(2-hydroxy-ethoxy)-ethylamine; 3-diethylaminopropylamine; 3-hydroxypropylamine; 6-hydroxyhexylamine; 3-imidazol-1-yl-propylamine; 2-(4-sulfamoylphenyl)ethylamine; 3-(4-methylpiperazin-1-yl)propylamine; 2-dimethylamino-1-methylethylamine; 2-[bis-(2-hydroxyethyl)amino] ethylamine; 1-carbamoyl-2-phenylethylamine; 2-dibutylaminoethylamine; 5-hydroxy-4,4-dimethylpentylamine; 3-dimethylamino-2,2-dimethylpropylamine; 2-(butylethylamino) ethylamine; 2-diisobutylaminoethylamine; 2-hydroxybutylamine; 3-hydroxy-2,2-dimethyl-propylamine; cyclohexylamine; (5-hydroxy-1,3,3-trimethy-cyclohexylmethyl)amine, 1,2,3,4-tetrahydronaphthalen-1-ylamine; cyclooctylamine; 3-(2-oxo-pyrrolidin-1-yl)propylamine; indan-1-ylamine; (tetrahydrofuran-2-ylmethyl-amine; 2-(lh-indol-3-yl)ethylamine; (benzo[1,3]dioxol-5-ylmethyl)amine; 3-morpholin-4-yl-propylamine; 2-pyridin-2-yl-ethylamine; 2-hydroxy-1-methyl-2-phenylethylamine; 1-methoxyethylamine; 1-methyl-3-phenypropylamine; 3-diethylamino-1-methylpropylamine; benzylamine; 2-fluorobenzylamine; 2-methoxy-benzylamine; 3-trifluoromethylbenzylamine; 2-phenylaminoethylamine; 2-methoxy-ethylamine; phenethylamine; 2-(2-methoxyphenyl)ethylamine; 2-(3,4-dimethoxy-phenyl)ethylamine; 2-(4-chlorophenyl)ethylamine; 2-(4-methoxyphenyl)ethylamine; 2-(4-hydroxyphenyl)ethylamine; 3,3-diphenylpropylamine; 2,5-dimethylbenzylamine; 2-trifluoromethylbenzylamine; butylamine; 1,2-diethylpyrazolidin-4-ylamine; 3-methoxypropylamine; 2-diisopropylaminoethylamine; 1-isopropyl-2-methylpropylamine; 3-m-tolylaminopentylamine; 3-butoxypropylamine; 1-(4-fluorophenyl)ethylamine; 1-methoxymethyl-propylamine; 2,3-dimethoxybenzylamine; 2,4-dimethoxybenzylamine; 2-(2-chloro-6-fluorobenzylsulfanyl)-ethylamine; 2,6-dimethoxy-benzylamine; 3,5-dimethoxybenzylamine; 2-phenoxyethylamine; 1-benzylpyrrolidin-3-ylamine; 2-(2,3-dimethoxyphenyl)ethylamine; 2-(2,5-dimethoxyphenyl)ethylamine; 2-(2-ethoxyphenyl) ethylamine; 2-(3,5-dimethoxyphenyl) ethylamine; 2-(4-ethoxyphenyl)ethylamine; 2-(4-trifluoromethoxyphenyl)ethylamine; 2-hydroxy-1,2-diphenylethylamine; 2-hydroxy-1,2-diphenylethylamine; 2-(2-hydroxymethylphenylsulfanyl)-benzylamine, 2-(3-fluorophenyl)ethylamine; 2-(2-aminophenyl)benzylamine, 2-(2-fluorophenyl)ethylamine; 4-aminobenzylamine; 2-(3,4-dimethoxyphenyl)ethylamine; 1,2-dihydroxy-2-(4-methylsulfanylphenyl)ethylamine; 2-hydroxycyclohexylamine; 3-(methylphenylamino)-propylamine; piperonyl; phenyl-N-methylpropyl; hydroxyethyloxyethyl; 2-indolinyl ethyl; and benzyl.

### EXAMPLES

The invention is further illustrated by way of the following examples which are intended to enable those skilled in the art to more clearly understand and to practice the present invention. These examples illustrate the synthesis of standards that were prepared using the method of the present invention. These examples are not intended, nor are they to be construed, as limiting the scope of the invention, but are merely illustrative and representative thereof.

### Example 1

### Synthesis of Standard A

Standard A, having the following formula was synthesized by the method described in Scheme I:

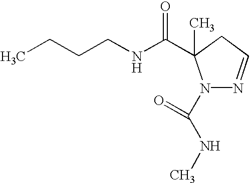

Molecular Formula: C₁₁H₂₀N₄O₂; Physical Properties: clear, colorless oil

¹H NMR (270 MHz, CDCl₃) δ: 8.55 (s, 1 H), 6.73 (s, 1 H), 6.07 (bs, 1 H), 3.89 (dd, J=19.3, 1.7 Hz, 1 H), 3.30-3.10 (m, 2 H), 2.85 (d, J=4.7 Hz, 3 H), 2.74 (dd, J=19.1, 1.7 Hz, 1 H), 1.66 (s, 3 H), 1.54-1.42 (m, 2 H), 1.39-1.22 (m, 2 H), 0.89 (dd, J=7.2, 6.5 Hz, 3 Hz)

¹³C NMR (67.5 MHz, CDCl₃) δ: 172.6, 156.9, 143.9, 68.4, 47.3, 39.6, 31.3, 26.7, 23.8, 20.1, 13.7

MS (ESI) m/z 241 [(M+H)⁺].

### Example 2

### Synthesis of Standard B

Standard B, having the following formula was synthesized by the method described in Scheme I:

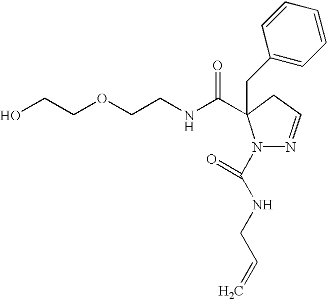

Molecular Formula: C₁₉H₂₆N₄O₄; Physical Properties: clear, colorless oil

¹HNMR (270 MHz, CDCl₃) δ: 9.41 (s, 1 H), 7.26-7.18 (m, 3 H), 7.13-7.06 (m, 2 H), 6.42 (bs, 1H), 6.29 (bt, J=5.8 Hz, 1H), 5.99-5.85 (m, 1H), 5.27-5.14 (m, 2 H), 4.11-3.99 (m, 1H), 3.93-3.88 (m, 1H), 3.85 (bs, 2 H), 3.81-3.73 (m, 2 H), 3.64-3.53 (m, 2 H), 3.47-3.35 (m, 1 H), 3.24 (bs,1 H), 3.09 (d, J=1.7 Hz, 1 H), 3.01 (s, 2 H), 2.97 (s, 2 H)

¹³C NMR (67.5 MHz, CDCl₃) δ: 171.9, 156.4, 144.2, 134.7, 134.3, 130.1, 128.2, 126.9, 115.8, 99.7, 76.3, 72.5, 72.3, 68.7, 61.5, 42.9, 42.3, 40.0, 39.7

MS (ESI) m/z 375 [(M+H)⁺].

### Example 3

### Synthesis of Standard C

Standard C, having the following formula was synthesized by the method described in Scheme I:

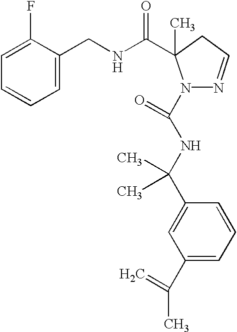

Molecular Formula: C₂₅H₂₉FN₄O₂; Physical Properties: clear, colorless oil

¹H NMR (270 MHz, CDCl₃) δ: 8.85 (bs, 1 H), 7.46 (s, 1 H), 7.32-7.13 (m, 5 H), 7.06-6.94 (m, 2 H), 6.80 (dd, J=1.7, 1.5 Hz, 1 H), 6.63 (bs, 1 H), 5.31 (s, 1 H), 5.05 (t, J=1.5 Hz, 1 H), 4.41 (d, J=3.7 Hz, 2 H), 3.86 (dd, J=19.0, 1.7 Hz, 1 H), 2.81 (dd, J=19.3, 1.7 Hz, 1 H), 2.11 (s, 3 H), 1.70 (s, 3 H), 1.68 (s, 3 H), 1.68 (s, 3 H)

¹³C NMR (67.5 MHz, CDCl₃) δ:173.3, 155.1, 147.0, 143.8, 143.5, 141.3, 129.2, 129.0, 128.8, 128.2, 124.2, 124.1, 123.8, 123.7, 121.7, 115.3, 115.0, 112.4, 68.2, 55.9, 47.5, 37.8, 30.3, 29.5, 23.3, 21.8

MS (ESI) m/z 437 [(M+H)⁺].

### Example 4

### Synthesis of Standard D

Standard D, having the following formula was synthesized by the method described in Scheme I:

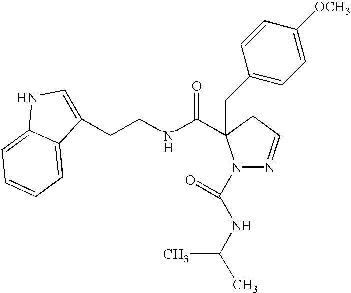

Molecular Formula: C₂₆H₃₁N₅O₃

¹H NMR (270 MHz, CDCl₃) δ: 8.98 (dd, J=5.2, 4.9 Hz, 1 H), 8.10 (bs, 1 H), 7.62 (d, J=7.4 Hz, 1 H), 7.34 (d, J=7.7 Hz, 1 H), 7.20-7.08 (m, 3 H), 6.95 (d, J=8.7 Hz, 2 H), 6.73 (d, J=8.6 Hz, 2 H), 6.38 (s, 1 H), 6.03 (bs, 1 H), 4.03-3.91 (m, 1 H), 3.75 (s, 2 H), 3.70-3.60 (m, 4 H), 3.55 (d, J=1.7 Hz, 1 H), 3.09-3.01 (m, 3 H), 2.93 (d, J=13.9 Hz, 1 H), 1.26 (d, J=6.5 Hz, 3 H), 1.18 (d, J=6.7 Hz, 3 H)

¹³C NMR (67.5 MHz, CDCl₃) δ: 173.2, 158.7, 155.9, 144.1, 136.3, 131.3, 131.3, 127.3, 126.6, 122.2, 122.0, 119.4, 118.7, 113.8, 112.7, 111.1, 72.2, 55.1, 43.7, 42.3, 40.8, 38.9, 24.9, 23.3, 23.0

MS (ESI) m/z 462 [(M+H)⁺].

### Example 5

### Synthesis of Standard E

Standard E, having the following formula was synthesized by the method described in Scheme I:

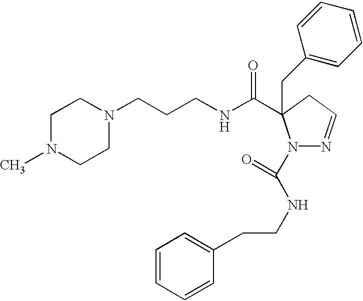

Molecular Formula: C₂₈H₃₈N₆O₂; Physical Properties: white solid

¹H NMR (270 MHz, CDCl₃) δ: 8.31 (dd, J=5.5, 5.4 Hz, 1 H), 7.34-7.16 (m, 8 H), 7.07-7.04 (m, 2 H), 6.28 (s, 1 H), 6.20 (t, J=5.7 Hz, 1 H), 3.73-3.30 (m, 17 H), 3.25-3.01 (m, 2 H), 2.93-2.83 (m, 2 H), 2.80 (s, 2 H), 2.27-2.21 (m, 1 H), 1.99-1.85 (m, 1 H)

MS (ESI) m/z 491 [(M+H)⁺].

### Example 6

### Synthesis of Standard F

Standard F, having the following formula was synthesized by the method described in Scheme I:

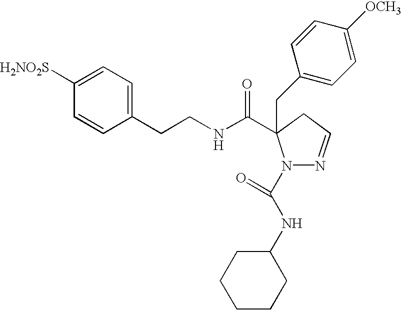

Molecular Formula: C₂₇H₃₅N₅O₅; Physical Properties: white solid

¹H NMR (270 MHz, CDCl₃) δ: 9.29 (t, J=5.4 Hz, 1 H), 7.81 (d, J=8.2 Hz, 2 H), 7.34 (d, J=8.4 Hz, 2 H), 6.97 (d, J=8.7 Hz, 2 H), 6.74 (d, J=8.6 Hz, 2 H), 6.06 (d, 8.2 Hz, 1 H), 5.00 (bs, 2 H), 3 .75 (s, 2 H), 3.72-3.62 (m, 2 H), 3.60-3.46 (m, 2 H), 3.02 (d, J=1.7 Hz, 1 H), 2.98-2.78 (m, 5 H), 2.10-1.87 (m, 2 H), 1.83-1.57 (m, 3 H), 1.52-1.12 (m, 6 H) ¹³C NMR (67.5 MHz, CDCl₃/CD₃OD) δ: 172.9, 158.1, 154.7, 151.1, 143.3, 142.6, 140.8, 130.6, 128.7, 126.3, 125.4, 113.0, 70.2, 54.0, 43.7, 40.0, 37.5, 34.5, 32.9, 32.4, 24.7, 24.2, 24.2

MS (ESI) m/z 542 [(M+H)⁺].

### Example 7

### Synthesis of Standard G

Standard G, having the following formula was synthesized by the method described in Scheme I:

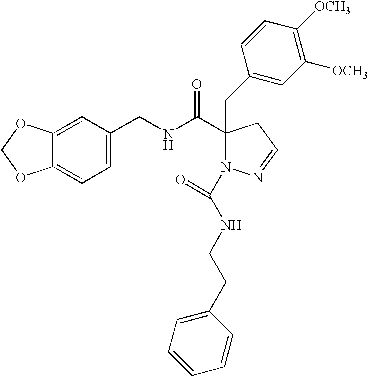

Molecular Formula: C₃₀H₃₂N₄O₆; Molecular Weight: 544.60; Physical Properties: yellow solid

¹H NMR (270 MHz, CDCl₃) δ9.28 (t, J=5.4 Hz, 1H), 7.33-7.18 (m, 5H), 6.81-6.72 (m, 5H), 6.62 (s, 1H), 6.40 (s, 1H), 6.25 (t, J =6.1 Hz, 1H), 5.90 (s, 2H), 4.4 (dq, J=17.4, 14.8 Hz, 2H), 3.82 (s, 3H), 3.78 (s, 3H), 3.72-3.60 (m, 2H), 3.51-3.41 (m, 2H), 3.02 (dd, J=19.3, 1.7 Hz, 1H), 2.95 (d, J=14 Hz, 1H), 2.85 (t, J=7.1 Hz, 2H)

¹³C NMR (67.5 MHz, CDCl₃) δ 172.2, 156.5, 148.6, 148.0, 147.7, 146.7, 144.2, 138.7, 132.3, 128.7, 128.6, 127.5, 126.5, 122.5, 120.7, 113.3, 110.9, 108.2, 108.1, 100.9, 72.3, 55.7, 55.6, 43.7, 42.9, 41.5, 39.5, 36.4

MS (ESI) m/z 545.3 [(M+H)⁺]

### Example 8

### Synthesis of Standard H

Standard H, having the following formula was synthesized by the method described in Scheme I:

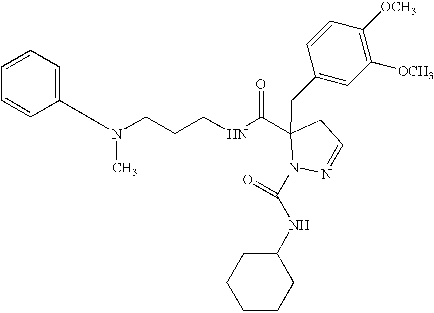

Molecular Formula: C₃₀H₄₁N₅O₄; Molecular Weight: 535.68; Physical Properties: yellow solid

¹H NMR (270 MHz, CDCl₃) δ 9.16 (s, 1H), 7.22 (t, J=8.0 Hz, 3H), 6.79-6.52 (m, 5H), 6.42 (t, J=1.5 Hz, 1H), 6.10 (d, J=8.2 Hz, 1H), 3.85 (s, 3H), 3.83 (s, 3H), 3.88-3.69 (m, 3H), 3.48-3.31 (m, 4H), 3.10 (dd, J=18.3, 1.7 Hz, 1H), 2.95 (s, 3H), 2.90 (d, J =14.3 Hz, 1H), 2.1-1.3 (m, 12H)

¹³C NMR (67.5 MHz, CDCl₃) 1672.4, 155.9, 148.8, 148.6, 148.0, 143.8, 129.1, 127.7, 122.5, 113.3, 112.3, 110.8, 72.3, 55.8, 55.7, 50.3, 49.1, 42.8, 39.5, 38.4, 37.7, 33.7, 33.4, 26.4, 25.4, 24.8

MS (ESI) m/z 536.2 [(M+H)⁺]

### Example 9

### Synthesis of Standard I

Standard I, having the following formula was synthesized by the method described in Scheme I:

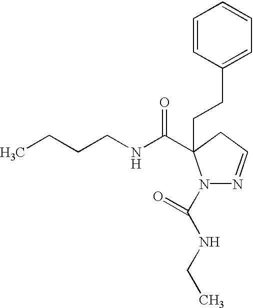

Molecular Formula: C₁₉H₂₈N₄O₂; Molecular Weight: 344.45; Physical Properties: clear, colorless oil

¹H NMR (270 MHz, CDCl₃) δ9.00 (s, 1H), 7.26-7.12 (m, 5H), 6.77 (t, J =1.7 Hz, 1H), 6.15 (t, J=5.5 Hz, 1H), 4.09 (dd, J=17.5, 1.8 Hz, 1H), 3.35-3.30 (m, 4H), 2.90 (dd, J=17.5, 1.8 Hz, 1H), 2.53-2.42 (m, 4 H), 1.51-1.48 (m, 2H), 1.36-1.32 (m, 2H), 1.97 (t, J=7.4 Hz, 3H), 0.91 (t, J=7.3 Hz, 3H)

¹³C NMR (67.5 MHz, CDCl₃) δ 171.8, 156.3, 143.9, 140.6, 128.4, 128.3, 126.1, 71.9, 43.7, 39.6, 37.8, 35.0, 31.3, 30.2, 20.1, 15.3, 13.7

MS (ESI) m/z 344.9 [(M+H)⁺]

### Example 10

### Synthesis of Standard J

Standard J, having the following formula was synthesized by the method described in Scheme I:

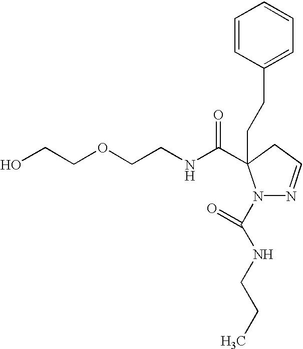

Molecular Formula: C₂₀H₃₀N₄O₄; Molecular Weight: 390.48; Physical Properties: white solid

¹H NMR (270 MHz, CDCl₃) δ 9.42 (s, 1H), 7.26-7.10 (m, 3H), 6.86 (s, 2H), 6.27 (t, J=5.7 Hz, 1H), 4.04 (dd, J=17.8, 1.7 Hz, 1H), 3.75-3.10 (m, 12H), 2.93 (dd, J=18.6, 1.7 Hz, 1H), 2.65-2.18 (m, 4H), 1.56 (q, J=7.2 Hz, 2H), 0.95 (t, J=7.4 Hz, 3H)

¹³C NMR (67.5 MHz, CDCl₃) δ 176.2, 160.9, 148.4, 144.7, 132.7, 132.6, 130.4, 77.0, 76.2, 73.1, 65.9, 48.1, 46.1, 44.0, 42.0, 34.3, 27.5, 15.5

MS (ESI) m/z 391.1 [(M+H)⁺]

### Example 11

### Synthesis of Standard K

Standard K, having the following formula was synthesized by the method described in Scheme I:

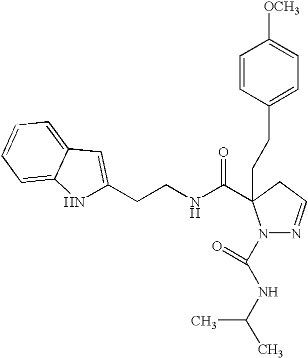

Molecular Formula: C₂₇H₃₃N₅O₃; Molecular Weight: 475.58; Physical Properties: orange solid

¹H NMR (270 MHz, CDCl₃) δ 9.0 (t, J=5.3 Hz, 1H), 8.34 (s, 1H), 7.61 (d, J=7.6 Hz, 1H), 7.29 (d, J=7.9 Hz, 1H), 7.17-6.71 (m, 8H), 6.01 (d, J=7.7 Hz, 1H), 4.12-3.45 (m, 3H), 3.80 (s, 3H), 3.15-2.85 (m, 4H), 2.56-2.29 (m, 4H), 1.20 (d, J=4.4 Hz, 3H), 1.18 (d, J=4.4 Hz, 3H)

¹³C NMR (67.5 MHz, CDCl₃) δ 172.1, 157.8, 155.5, 143.7, 136.2, 132.5, 129.2, 127.3, 121.9, 121.7, 119.1, 118.6, 113.7, 112.9, 111.0, 71.7, 55.1, 43.8, 42.1, 40.4, 37.9, 29.1, 25.2, 23.2, 22.9

MS (ESI) m/z 476.2 [(M+H) +]

### Example 12

### Synthesis of Standard L

Standard L, having the following formula was synthesized by the method described in Scheme I:

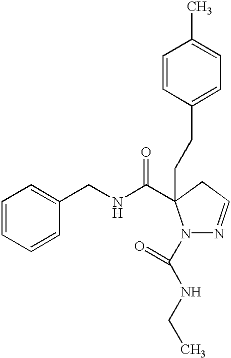

Molecular Formula: C₂₃H₂₈N₄O₃; Molecular Weight: 408.49; Physical Properties: clear, colorless oil

¹H NMR (270 MHz, CDCl₃) δ 9.44 (t, J=4.9 Hz, 1H), 7.32-7.21 (m, 5H), 7.00 (d, J=8.6 Hz, 2H), 6.78 (d, J=8.6 Hz, 2H), 6.75 (s, 1H), 6.14 (t, J=5.5 Hz, 1H), 4.44 (dq, J=25.4, 14.9 Hz, 2H), 4.05 (dd, J=17.6, 1.7 Hz, 1H), 3.75 (s, 3H), 3.34-3.24 (m, 2H), 2.92 (dd, J=17.8, 1.7 Hz, 1H), 2.56-2.32 (m, 4H), 1.15 (t, J =7.18, 3H)

¹³C NMR (67.5 MHz, CDCl₃) δ 172.0, 157.9, 156.3, 143.9, 138.5, 132.6, 129.2, 128.5, 127.3, 127.0, 113.8, 71.9, 55.2, 43.8, 43.7, 37.9, 35.0, 29.2, 15.3

MS (ESI) m/z 409.0 [(M+H) +]

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, method, process step or steps, to the objective, spirit and scope of the present invention. All such modifications are intended to be within the scope of the claims appended hereto.

We claim:

1. A method for the synthesis of a compound or an array of compounds of Formula I: 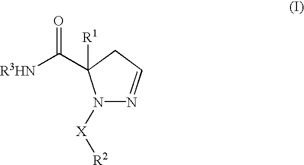
wherein:
R¹ is selected from the group consisting of C₁₋₁₄ alkyl; (CH₂)_{0- 6}aryl; (CH₂)₀₋₂CH(Ph)₂; (CH₂)1₄aryl(C₁₋₄alkoxy)₁₋₃; (CH₂)₀₋₄-Z-(CH₂)₁₋₄aryl(C₁₋₄alkoxy)₀₋₄; (CH₂)₁₋₄-Z-aryl-(CF₃)₁₋₂; and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl;
R² is selected from the group consisting of H, C₁₋₁₄ straight chain or branched alkyl; C₄₋₁₀cycloalkyl; (CH₂)₁₋₄aryl; 1-(3-isopropenylphenyl)-1-methylethyl; phenylethyl; 1,3-dioxo-1,3-dihydroisoindol-2-ylmethyl and C₂₋₈alkenyl;
R³ represents a suitable primary amine substituent; and
X is selected from the group consisting of a covalent bond, CONH and CO, with the proviso that when X is a covalent bond, R² is H;
said method comprising the steps of:
(i) treating in an inert solvent, a compound of Formula 1:
SS—OH (1)
with a compound or an array of compounds of Formula 2: 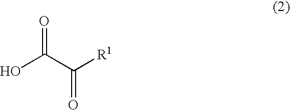
in the presence of a dehydrating agent and an optional catalyst, to form a compound or an array of compounds of Formula 3: 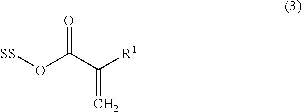
where SS represents a solid support and R¹ is as defined above;
(ii) treating an inert solvent mixture of a compound or an array of compounds of Formula 3 with a compound of Formula 4:
PG—CH—N₂ (4)
to form a compound or an array of compounds of Formula 5: 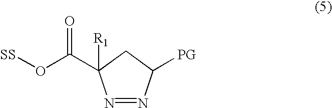
where PG represents a protecting group and SS and R¹ are as defined above;
(iii) treating an inert solvent solution of a compound of Formula 5 with a deprotecting agent to form a compound of Formula 6: 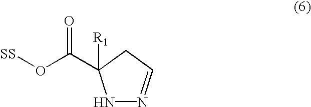
where SS and R¹ are as defined above;
(iv) optionally treating an inert solvent mixture of a compound or an array of compounds of Formula 6 with a compound or an array of compounds of Formula 7:
R²—W (7)
to form a compound or an array of compounds of Formula 8: 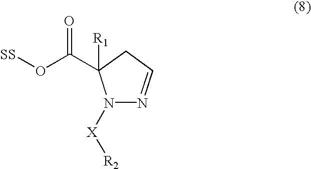
where W represents —COCl, —N═C═O or —COOH; and SS, R¹, R² and X are as defined above;
(v) treating a solution of a compound or an array of compounds of Formula 6 or Formula 8 with a compound or an array of compounds of Formula 9:
R³—NH₂ (9)
in the presence of an optional base to form a compound of Formula I.

2. The method of claim 1 wherein a compound or an array of compounds of Formula 6 is treated with a compound or an array of compounds of Formula 7 to form a compound or an array of compounds of Formula 8.

3. The method of claim 2 wherein the base in step (i) is a tertiary amine.

4. The method of claim 3 wherein the tertiary amine is selected from the group consisting of diisopropylethylamine, triethylamine, N-methylmorpholine and lutidine.

5. The method of claim 4 wherein the inert solvent in step (ii) is selected from the group consisting of toluene, tetrahydrofuran, hexanes and CH₂Cl₂.

6. The method of claim 5 wherein the compound of Formula 4 in step (ii) is trimethylsilyl diazomethane.

7. The method of claim 6 wherein the deprotecting agent in step (iv) is selected from the group consisting of trifluoroacetic acid and silver triflate.

8. The method of claim 7 wherein the compound of Formula 7 in step (iv) is an isocyanate represented by R²—N═C═O.

9. The method of claim 8 wherein step (iv) utilizes a solution of a compound of Formula 8 in pyridine, tetrahydrofuran, dioxane or mixtures thereof.

10. The method of claim 9 wherein step (v) is carried out in the presence of a base selected from the group consisting of triethylamine and diisopropyl ethyl amine.

11. The method of claim 10 wherein:
R¹ is selected from the group consisting of C₁₋₈alkyl; (CH₂)₁₋₂-Ph; 4-methoxybenzyl; 4-methoxyphenylethyl; 3,4-dimethoxybenzyl; 2-benzyloxyethyl; 2-benzyloxypropyl; 3-methoxy-2-benzyloxyethyl; and 3-trifloromethyl-2-benzyloxyethyl;
R² is selected from the group consisting of methyl, ethyl, i-propyl, propyl, t-butyl, cyclohexyl, phenylethyl, benzyl, CH₂—CH═CH₂, 1-(3-isopropenylphenyl)-1-methylethyl and 1,3-dioxo-1,3-dihydroisoindol-2-ylmethyl; and
R³ is selected from the group consisting of 2-hydroxy-1-hydroxymethyl-2-phenylethyl; 2-hydroxy-1-hydroxymethyl-3-methylpentyl; 1-benzyl-2-hydroxyethyl; 1-hydroxymethyl-3-methylbutyl; 4-amino-piperidine-1-carboxylic acid ethyl ester; 2-acetylaminoethyl; 2-diethylaminoethyl; 2-(2-hydroxyethoxy)ethyl; 3-diethylaminopropyl; 3-hydroxypropyl; 6-hydroxyhexyl; 3-imidazol-1-yl-propyl; 2-(4-sulfamoylphenyl)ethyl; 3-(4-methylpiperazin-1-yl)-propyl; 2-dimethylamino-1-methylethyl; 2-[bis-(2-hydroxy-ethyl)amino]ethyl; 1-carbamoyl-2-phenylethyl; 2-dibutylaminoethyl; 5-hydroxy-4,4-dimethylpentyl; 3-dimethylamino-2,2-dimethylpropyl; 2-(butylethylamino)ethyl; 2-diisobutylaminoethyl; 2-hydroxybutylamino; 3-hydroxy-2,2-dimethylpropyl; cyclohexyl; (5-hydroxy-1,3,3-trimethylcyclohexylmethyl); 1,2,3,4-tetrahydronaphthalen-1-yl; cyclooctyl; 3-(2-oxopyrrolidin-1-yl)-propyl; indan-1-yl; (tetrahydrofuran-2-ylmethyl)-; 2-(1 h-indol-3-yl)-ethyl; (benzo[1,3]dioxol-5-ylmethyl); 3-morpholin-4-yl-propyl; 2-pyridin-2-yl-ethyl; 2-hydroxy-1-methyl-2-phenylethyl; 1-methoxyethyl; 1-methyl-3-phenylpropyl; 3-diethyl-1-methylpropyl; benzyl; 2-fluorobenzyl; 2-methoxybenzyl; 3-trifluoromethylbenzyl; 2-phenylaminoethyl; 2-methoxyethyl; phenylethyl; 2-(2-methoxyphenyl)ethyl; 2-(3,4-dimethoxyphenyl)ethyl; 2-(4-chlorophenyl)ethyl; 2-(4-methoxy-phenyl)ethyl; 2-(4-hydroxyphenyl)ethyl; 3,3-diphenylpropyl; 2,5-dimethylbenzyl; 2-trifluoromethylbenzyl; butyl; 1,2-diethyl-pyrazolidin-4-yl; 3-methoxypropyl; 2-diisopropylaminoethyl; 1-isopropyl-2-methylpropyl; 3-m-tolylaminopentyl; 3-butoxypropyl; 1-(4-fluorophenyl)ethyl; 1-methoxymethylpropyl; 2,3-dimethoxybenzyl; 2,4-dimethoxybenzyl; 2-(2-chloro-6-fluorobenzylsulfanyl)ethyl; 2,6-dimethoxybenzyl; 3,5-dimethoxybenzyl; 2-phenoxyethyl; 1-benzylpyrrolidin-3-yl; 2-(2,3-dimethoxyphenyl)ethyl; 2-(2,5-dimethoxyphenyl)ethyl; 2-(2-ethoxyphenyl)ethyl; 2-(3,5-dimethoxyphenyl)ethyl; 2-(4-ethoxyphenyl)ethyl; 2-(4-trifluoromethoxyphenyl)ethyl; 2-hydroxy-1,2-diphenylethyl; 2-hydroxy-1,2-diphenylethyl; 2-(2-hydroxymethylphenylsulfanyl) benzyl, 2-(3-fluorophenyl)ethyl; 2-(2-aminophenyl)benzyl; 2-(2-fluorophenyl)ethyl; 4-aminobenzyl; 2-(3,4-dimethoxyphenyl)ethyl; 1,2-dihydroxy-2-(4-methylsulfanylphenyl)ethyl; 2-hydroxycyclohexyl; 3-(methylphenylamino)propyl; piperonyl; phenyl-N-methylpropyl; hydroxyethyloxyethyl; 2-indolinyl ethyl; and benzyl.

12. A method for synthesizing a compound or an array of compounds of Formula 5: 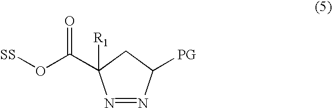
wherein:
PG represents a protecting group;
S represents a solid support; and
R¹ is selected from the group consisting of C₁₋₁₄alkyl; (CH₂)₀₋₆aryl; (CH₂)0-2CH(Ph)₂; (CH₂)₁₋₄aryl-(C₁₋₄alkoxy)1-3; (CH₂)₀₋₄-Z-(CH₂)₁₋₄aryl(C₁₋₄alkoxy)₀₋₄; (CH₂)₁₋₄-Z-aryl-(CF₃)₁₋₂; and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl;
said method comprising treating an inert solvent mixture of a compound or an array of compounds of Formula 3 with a compound of Formula 4:
PG—CH—N₂ (4)
to form a compound or an array of compounds of Formula 5.

13. A method for synthesizing a compound or an array of compounds of Formula 6: 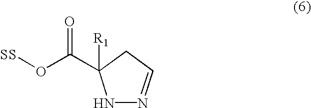
wherein:
SS represents a solid support; and
R¹ is selected from the group consisting of C₁₋₁₄alkyl, (CH₂)₀₋₆aryl, (CH₂)₀₋₂CH(Ph)₂, (CH₂)₁₋₄aryl(C₁₋₄ alkoxy)₁₋₃, (CH₂)₀₋₄-Z-(CH₂)₁₋₄aryl (C₁₋₄alkoxy)₀₋₄, (CH₂)₁₋₄-Z-aryl-(CF₃)₁₋₂, and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl;
said method comprising treating an inert solvent solution of a compound of Formula 5: 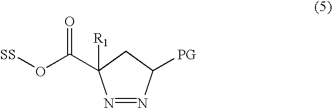
with a deprotecting agent to form a compound of Formula 6, where PG represents a protecting group.

14. A method for synthesizing a compound or an array of compounds of Formula 8: 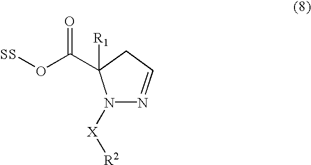
wherein:
SS represents a solid support;
R¹ is selected from the group consisting of C₁₋₁₄alkyl; (CH₂)0-6aryl; (CH₂)₀₋₂CH(Ph)₂; (CH₂)₁₋₄aryl(C₁₋₄alkoxy)₁₋₃; (CH₂)₀₋₄-Z-(CH₂)₁₋₄aryl(C₁₋₄alkoxy)₀₋₄; (CH₂)₁₋₄-Z-aryl-(CF₃)₁₋₂; and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl;
R² is selected from the group consisting of H, C₁₋₁₄ straight chain or branched alkyl; C₄₋₁₀cycloalkyl; (CH₂)₁₋₄aryl; 1-(3-isopropenylphenyl)-1-methylethyl; phenylethyl; 1,3-dioxo-1,3-dihydroisoindol-2-ylmethyl and C₂₋₈alkenyl; and
X is selected from the group consisting of a covalent bond, CONH and CO, with the proviso that when X is a covalent bond, R² is H;
said method comprising treating an inert solvent mixture of a compound or an array of compounds of Formula 6: 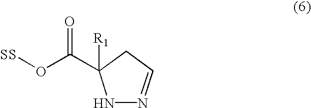
with a compound or an array of compounds of Formula 7:
R²—W (7)
where W represents —COCl, —N═C═O or —COOH, to form a compound or an array of compounds of Formula 8.

15. A compound of Formula 5: 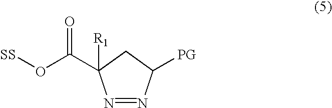
wherein:
SS represents a solid support;
PG represents a protecting group; and
R¹ is selected from the group consisting of C₁₋₁₄alkyl; (CH₂)0-6aryl; (CH₂)₀₋₂CH(Ph)₂; (CH₂)₁₋₄aryl(C₁₋₄alkoxy),-₃; (CH₂)₀₋₄-Z-(CH₂)₁₋₄aryl(C₁₋₄alkoxy)₀₋₄; (CH₂)₁₋₄-Z-aryl-(CF₃)₁₋₂; and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl.

16. A compound of Formula 6: 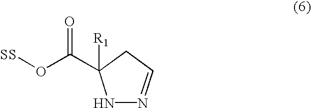
wherein:
SS represents a solid support; and
R¹ is selected from the group consisting of C₁₋₁₄alkyl; (CH₂)₀₋₆aryl; (CH₂)0-2CH(Ph)₂; (CH₂)₁₋₄aryl(C₁₋₄alkoxy)₁₋₃; (CH₂)₀₋₄-Z-(CH₂)₁₋₄aryl(C₁₋₄alkoxy)₀₋₄; (CH₂)₁₋₄-Z-aryl-(CF₃)₁₋₂; and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl.

17. A compound of Formula 8: 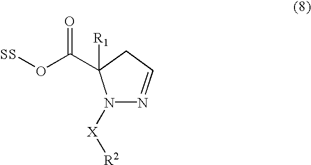
wherein:
SS represents a solid support;
R¹ is selected from the group consisting of C₁₋₁₄alkyl; (CH₂)₀₋₆aryl; (CH₂)₀₋₂CH(Ph)₂; (CH₂)₁₋₄aryl(C₁₋₄alkoxy)₁₋₃; (CH₂)₀₋₄-Z-(CH₂)1-4aryl(C₁₋₄alkoxy)₀₋₄; (CH₂)₁₋₄-Z-aryl-(CF₃)₁₋₂; and C₂₋₁₄alkenyl substituted with up to three substituents selected from the group consisting of Ph and C₁₋₄alkyl, where Z is selected from the group consisting of O, S and NR⁴ where R⁴ is C₁₋₁₄alkyl or aryl;
R² is selected from the group consisting of H, C₁₋₁₄ straight chain or branched alkyl; C₄₋₁₀cycloalkyl; (CH₂)₁₋₄aryl; 1-(3-isopropenylphenyl)-1-methylethyl; phenylethyl; 1,3-dioxo-1,3-dihydroisoindol-2-ylmethyl and C₂₋₈alkenyl; and
X is selected from the group consisting of a covalent bond, CONH and CO, with the proviso that when X is a covalent bond, R² is H.
